(19) United States
(12) Patent Application Publication
(10) Pub. No.: US 2006/0121620 A1
(43) Pub. Date: Jun. 8, 2006

(54) Nitrate/nitrite assay reagents, kit, and method of use

(76) Inventor: Basant Bhandari, San Antonio, TX (US)

Correspondence Address: INTELLECTUAL PROPERTY / TECHNOLOGY LAW, PO BOX 14329, RESEARCH TRIANGLE PARK, NC, 27709, US

(21) Appl. No.: 11/005,516

(22) Filed: Dec. 6, 2004

(51) Int. Cl. G01N 33/00

(52) U.S. Cl. 436/110; 422/61

(57) ABSTRACT

An assay kit and method for the quantitative and direct determination of the concentration of nitrate and nitrite ions in a sample solution. The nitrate assay is based on the reaction of methyl salicylate with nitrate ions to form 5-nitro-2-hydroxymethylbenzoic acid, which has a yellow color having a maximum absorbance of λ=410 nm. The nitrite assay is a derivative of the well-known Greiss diazotization reaction.

### BACKGROUND OF THE INVENTION

1. Field of the Invention

The present invention relates generally to reagents, a kit and method for quantitatively and directly determining the concentration of nitrate and nitrite ions in a sample solution.

2. Description of the Related Art

Nitrate ions from fertilizers, treated sewage and industrial processes have reached disquietingly high concentrations in water supplies all around the world and many have reported that elevated nitrate concentrations may be the cause of several maladies. For example, it has been suggested that increased nitrate levels cause methemoglobinemia, more commonly referred to as “blue-baby syndrome.” In addition, a recent study by the University of Iowa has shown a link between nitrate levels in drinking water and bladder cancer in women (see, Weyer, P. J., Cerhan, J. R., Kross, B. C., Hallberg, G. R., Kantamneni, J. Breuer, G., Jones, M. P., Zheng, W., Lynch, C. F., Epidemiology, 12(3), 327-28 (2001)). In response, the United States Environmental Protection Agency (EPA) has fixed an allowable upper limit of 10 ppm for NO₃⁻-nitrogen (NO₃⁻N) in drinking water.

Excess nitrate in water also has a detrimental effect on ecosystems. When a nitrogen limited ecosystem is supplied with high levels of nitrate, significant increases in the levels of phytoplankton (algae) and macrophytes (aquatic plants) can occur, i.e., eutrophication, which can pose a threat to fragile ecosystems. It has been reported that to avoid the propagation of algal blooms, the nitrate concentration should be between 0.1 to 1 mg/L (NOAA/EPA).

Evidence that nitrates are harmful to humans, other animals and the ecosystem prompts a continuing need to monitor nitrates in drinking water, watersheds, industrial wastewater, private wells and estuaries. Additionally, nitrate contamination of source water is a concern for industries that depend on water purity during the manufacturing of their finished product.

Nitric oxide (NO) is produced by a variety of mammalian cells and tissues. Nitric oxide is synthesized from the amino acid L-arginine by the nitric oxide synthase (NOS). So far, the only clearly established role for NO is as a cytotoxic molecule for invading microorganisms and tumor cells. However, other physiological activity, such as acting as a neurotransmitter in the brain and in the periphery, affecting gastrointestinal tract motility and penile erection were also observed. Nitric oxide is produced in vascular endothelial cells by the NOS and seems to mediate vascular smooth muscle relaxation by increasing levels of cGMP.

In vivo, NO is metabolized to the stable products nitrate and nitrite, which may subsequently be assayed in urine, plasma, tissue, or other specimens. As such, the level of nitrate or nitrite in a specimen may serve as an indicator of the level of NO synthesis in a patient and may be suitable for detection, diagnosis and prediction of diseases including, but not limited to, cancer, AIDS, rheumatoid arthritis, and diabetes. For example, it has been reported that NO participates in the multi-step process of carcinogenesis. Nitric oxide also plays a major role in altering glucose transport in different types of cells and tissues. Additionally, preterm labor may be predicted by detecting levels of NO in myometrium using non-invasive, non-surgical methods for detection of NO in blood, urine, saliva and tissue samples.

Recent research on the role of nitric oxide (NO) in wound inflammation, tissue repair, and microvascular homeostasis has allowed us to consider NO as a primary regulator of wound healing (Bruch-Gerharz, D., Ruzicka, T., Kolb-Bachofen, V., J Invest. Dermatol., 110(1), 1-7 (1998); Schaffer, M. R., Tantry, U., Efron, P. A., Ahrendt, G. M., Thornton, F. J., Barbul, A., Surgery, 121(5), 513-9 (1997)). A systemic deficiency of endothelial-derived NO has been observed in all diabetics, suggesting that NO plays a fundamental role in the pathogenesis of chronic, non-healing lower extremity ulceration (LEU), which is associated with significant morbidity and treatment costs (Veves, A., Akbari, C. M., Primavera, J., Donaghue, V. M., Zacharoulis, D., Chrzan, J. S., DeGirolami, U., LoGerfo, F. W., Freeman, R., Diabetes, 47, 457 (1998); Huszka, M., Kaplar, M., Rejto, L., Tomai, I., Palatka, K., Laszlo, P., Udvardy, M., Thrombosis Res., 86(2), 173 (1997); Williams, S. B., Cusco, J. A., Roddy, M. A., Hohnston, M. T., Creager, M. A., J. Am. Col. Cardiol., 27(3), 567 (1996)).

Nitrate in the presence of nitrite is usually estimated by reducing nitrate to nitrite using agents such as hydrazine (Kamphake, L. J., Hannal, S. A., Cohen, J. M., Water Res., 1, 205 (1967)), Cd—Cu (Wood, E. D., Armstrong, F. A. J., Richards, F. A., J. Mar. Biol. Assoc. (UK), 47, 23 (1967)) or nitrate reductase from E. coli (McNamara, A., Meeker, G. B., Shaw, P. D., Hageman, R. H., J. Agric. Food Chem., 19, 224 (1971)), and determining the total nitrite concentration (for example using the Greiss reaction). Direct estimation of nitrate concentrations may be determined by nitrating brucine or Xylen-1-ol (Nicholas, D. J. D., Nason, A., in Methods in Enzymology, Vol. III, edited by Colowick, S. P., Kaplan, N. O., Academic Press, New York, pg 981 (1957)). Disadvantages of these methods include stringent experimental conditions and tedious, time consuming assays.

Cataldo et al. previously reported that nitrate concentrations in plant tissue may be directly determined calorimetrically by nitrating salicylic acid (see, Cataldo, D. A., Haroon, M., Schrader, L. E., Youngs, V. L., Commun. Soil Sci. and Plant Analysis, 6(1), 71-80 (1975) (hereinafter Cataldo et al.)). Disadvantages of the Cataldo et al. method include large measurement volumes which must be disposed of, lack of sensitivity at low nitrate concentrations, the weighing of small quantities of salicylic acid for every sample and standard solution, lack of reproducibility, and difficulty in rapidly measuring a large number of samples at once.

A method for the determination of nitrates using sodium salicylate has also been described (Lange/Vejdelek, Photometrische Analyse (Photometric Analysis), Verlag Chemie Weinheim, 7th Edition, 1980, page 365), with the yellow 5-nitrosalicylic acid obtained being determined by photometry. Using this method, it is necessary to remove interfering anions with an ion exchanger prior to analysis, since nitrites increase the intensity and bromides and iodides reduce the intensity of the color obtained. Moreover, the determination is very elaborate, slow and tedious. The sample containing the reagent must be evaporated to dryness by heating at 100°-120° C. After cooling, 96% sulfuric acid is added to the dried residue. Then, only after the addition of water and alkali solution and standing for 20 minutes, can the absorbance be measured.

Methods proposed for the simultaneous determination of nitrate and nitrite ions have been reported, however, to date none have efficiently and reproducibly disclosed the direct determination of nitrate and nitrite concentrations in a portable, inexpensive, sensitive, easy-to-use kit. For example, electromigration and chromatographic methods are currently being used to simultaneously determine these species in water, however, the instrumentation is expensive and often preconcentration and/or separation steps are required prior to species analysis. Moreover, these methods do not specifically provide the concentration of nitrate relative to nitrite but rather the cumulative amount of nitrate plus nitrite ions following reduction and measurement of the total nitrite concentration.

It would therefore be a significant advance in the art of nitrate and nitrite ion determination to provide improved reagents, kits and methods that overcome the disadvantages of the prior art. Specifically, it would be a significant advance in the arts to provide a rapid, reproducible, and sensitive micro-sampling method for the direct determination of nitrate ions in a sample solution.

In addition, it would be a significant advance in the art to assay body fluids for the stable NO metabolites nitrate and nitrite ions to detect, diagnose and predict diseases such as cancer and diabetes.

### SUMMARY OF THE INVENTION

The present invention relates generally to reagents, a kit and method for quantitatively and directly determining the concentration of nitrate and nitrite ions in a sample solution. Specifically, the present invention relates to a micro-sampling kit and method that is rapid, reproducible and sensitive, as well as rugged and simple enough to quantitatively determine the concentration of nitrate ions regardless of the circumstances.

In one aspect, the present invention relates to an assay kit comprising reagents for conducting a nitrate assay, wherein when said reagents are combined with a nitrate-containing sample, a colorimetric change is provided which is detectable by spectrophotometry or colorimetry and enables the concentration of nitrate in the nitrate-containing sample to be directly determined.

In another aspect, the present invention relates to a method for directly determining the concentration of nitrate ions in a nitrate-containing sample, said method comprising:
(a) transferring an aliquot of the nitrate-containing sample to a tube;
(b) mixing an aliquot of sulfuric acid with an aliquot of a methyl salicylate solution to form an acidic methyl salicylate solution;
(c) adding an aliquot of the acidic methyl salicylate solution to the nitrate-containing sample to form an acidic reaction mixture;
(d) adding at least one aliquot of a sodium hydroxide solution to the acidic reaction mixture to form a basic reaction mixture;
(e) cooling the basic reaction mixture; and
(f) measuring the absorbance of the basic reaction mixture at λ=410 nm.

In a further aspect, the present invention relates to an assay kit comprising reagents for conducting a nitrate assay and a nitrite assay, wherein when said reagents are combined with a sample solution, a colorimetric change is provided which is detectable spectrophotometrically or colorimetry, and enables the concentration of nitrate and nitrite in a sample solution to be separately and directly determined.

Other aspects, features and embodiments of the invention will be more fully apparent from the ensuing disclosure and appended claims.

### BRIEF DESCRIPTION OF THE DRAWINGS

FIG. 1A is the standard absorbance curve for reaction mixtures having 0-300 nmoles of nitrate per 100 μL of solution, according to the present invention.

FIG. 1B is the standard absorbance curve for reaction mixtures having 0-50 nmoles of nitrate per 100 μL of solution, according to the present invention.

FIG. 2 is a bar graph comparing the absorbance of the nitrate-containing reaction mixture at λ=410 nm for varying concentrations of methyl salicylate reactant.

FIG. 3A is the standard absorbance curve for reaction mixtures having 0-100 μM nitrite, according to one embodiment of the nitrite assay of the present invention.

FIG. 3B is the standard absorbance curve for reaction mixtures having 0-20 nmoles of nitrite per 200 μL of solution, according to one embodiment of the nitrite assay of the present invention.

FIG. 4 is the standard absorbance curve for reaction mixtures having 0-30 μM nitrite, according to one embodiment of the nitrite assay of the present invention.

### DETAILED DESCRIPTION OF THE INVENTION, AND PREFERRED EMBODIMENTS THEREOF

There is a great need in the art for a simple, sensitive, reproducible micro-sampling method for the determination of nitrate ions in a sample solution. Towards that end, the present invention relates to reagents, a kit and micro-sampling method that is rugged and simple enough to quantitatively determine the concentration of nitrate ions regardless of the circumstances. The kit includes the reagents and instructions for nitrate analysis as described herein, and may optionally include the reagents and instructions for nitrite analysis as described herein.

The disclosure of the following publication is hereby incorporated by reference herein in its respective entirety: Bhandari, B., Simlot, M. M., “Rapid Micro-method for Determination of Nitrate in Presence of Nitrite for Biochemical Studies,” Ind. J. of Exp. Biol., 24, 323-325 (1986) (hereinafter “the Bhandari paper”).

The Bhandari paper, which was principally authored by the present inventor, relates to the nitration of salicylic acid in concentrated sulfuric acid to produce 5-nitro-2-hydroxybenzoic acid, and represents an improvement over the Cataldo et al. method. For example, smaller reagent volumes were used while the sensitivity of the analysis increased. The invention disclosed herein embodies various improvements to the nitrate assay disclosed in such Bhandari paper.

The methods disclosed in Cataldo et al. and the Bhandari paper utilized solid salicylic acid as the reagent to be nitrated in concentrated sulfuric acid. A disadvantage of these previous methodologies included the necessity of weighing a small amount of salicylic acid for each sample and standard solution to be measured, which is tedious and subject to error. Further, the salicylic acid was not readily soluble in concentrated sulfuric acid and the mixture had to be vortexed vigorously to effect dissolution.

Derivatives of salicylic acid are promising substitutes for salicylic acid for nitration. Specifically, methyl salicylate (2-hydroxybenzoic acid methyl ester, C₈H₈O₃) in the liquid form is a particularly promising candidate because it is pipettable and is readily miscible with concentrated sulfuric acid. Further, methyl salicylate is highly stable under recommended storage conditions.

As defined herein, the term “assay” is broadly defined and may represent the analysis of a substance to determine the concentration of components, the substance to be analyzed, or the result of such analysis.

In one embodiment, the method of the present invention for the direct determination of nitrate concentration comprises the reaction of methyl salicylate with nitrate ions to form 5-nitro-2-hydroxymethylbenzoic acid, which has a yellow color having a maximum absorbance of λ=410 nm.

In another embodiment, the nitrate determination method is provided in the form of a kit. Kit components include a methyl salicylate solution, at least one sodium hydroxide solution having a pH greater than 14, and instructions. Preferably, the concentration of methyl salicylate is in a range of about 0.5 vol. % to about 2.0 vol. %, based on the total volume of the solution. The kit may further comprise a second sodium hydroxide solution having a pH that is less than the pH of the first sodium hydroxide solution (i.e., the second NaOH solution is more dilute than the first NaOH solution). Preferably, the pH of the second NaOH solution is about 2 M to about 8 M and the pH of the first NaOH solution is about 6 M to about 12 M. The kit may optionally include sulfuric acid, preferably in a range from about 15 M to about 18 M, most preferably 18 M (i.e., concentrated H₂SO₄). The kit may further include a standard solution of sodium nitrate for preparation of a standard nitrate curve. Importantly, the volume of reagents provided in the kit is dependent on whether the absorbance measurement is performed using a well plate reader or a spectrophotometer/colorimeter (to be discussed at length hereinbelow).

For the determination of nitrate ions in mammalian cells, serum free media should be used. As such, the kit may include the standard solutions of nitrate in a serum free medium for preparation of the standard nitrate curve.

For example, the kit for 100 nitrate ion assays may comprise the reagents in the following quantities and concentrations:
1. Standard solution of sodium nitrate: 3 mL of 1 mM, and 0.5 mL of 10 mM;
2. Reagent A: 0.4 mL of 0.5 vol. % to 2 vol. % methyl salicylate, based on the total volume of the solution;
3. Reagent B: 50 mL 5 N sodium hydroxide in distilled water (preferably stored between 4° C. and 10° C.); and
4. Reagent C: 40 mL 10 N sodium hydroxide in distilled water (preferably stored between 4° C. and 10° C.).
   It is contemplated herein that the kit is provided without the more dilute NaOH (Reagent B) along with instructions to dilute Reagent C to form Reagent B. It is also contemplated that the standard solution of sodium nitrate will not be present in the kit, but the preparation of same will be provided in the instructions.

It is understood by one skilled in the art that the volume and/or concentration of the kit components may be altered as long as the ratios of components to one another are substantially similar to those disclosed herein. For example, the volumes of the kit components may increase or decrease depending on the number of assays desired. Alternatively, the concentration and volume of the kit components may be altered commensurate to one another to increase or decrease the volume of each individual assay.

In addition to the four kit components enumerated above, 20 mL of sulfuric acid is needed, which can be provided in the kit or must be purchased separately by the kit user. Prior to assaying, Reagent A must be reconstituting by combining 20 mL of concentrated H₂SO₄ with Reagent A, followed by vortexing to mix. The reconstituted Reagent A is preferably stored at about −20° C.

The instructions provided in the assay kit for the determination of nitrate ion concentration may read essentially as follows:
1. Transfer about 0-100 μL of a sample solution containing nitrate (0-300 nmole or 0-4.2 μg NO₃⁻—N) to a glass tube or other H₂SO₄ resistant tube, e.g., disposable cuvettes that do not interfere with absorbance measurements at λ=410 nm, having a volume capacity greater than 2 mL, e.g., 10-15 mL. Adjust the volume to 100 μL with distilled water, if necessary;
2. Add about 200 μL of Reagent A to the tube and vortex to mix. The time of vortexing may be from about 5 sec to about 30 sec, preferably about 10 sec. If cloudiness is observed, re-vortex until the opacity fades;
3. Add about 500 μL of Reagent B to the tube slowly and carefully vortex to mix. The time of vortexing may be from about 5 sec to about 30 sec, preferably about 10 sec. It is not abnormal for the solution to have some level of opaqueness;
4. Add about 400 μL of Reagent C to the tube slowly and carefully vortex to mix. The time of vortexing may be from about 5 sec to about 30 sec, preferably about 10 sec. If cloudiness is observed, re-vortex until the opacity fades;
5. Let the reaction mixture cool for about 5 to about 10 min; and
6. Transfer the reaction mixture to a glass cuvette and measure absorbance at λ=410 nm using a colorimeter or a spectrophotometer. Using Beer's Law and the standard nitrate curve, calculate the concentration of nitrate in the sample.

It is understood by one skilled in the art that the volume and/or concentration of the reagents used may be altered as long as the ratios of components to one another are substantially similar to those disclosed herein. For example, the volumes of the reagents used may increase or decrease depending on the amount of sample available or the degree of dilution. Alternatively, the concentration and volume of the reagents used may be altered commensurate to one another to increase or decrease the absorbance of the reaction mixture.

Although not wishing to be bound by theory, two aliquots of NaOH are added to the reaction mixture to neutralize the sulfuric acid to improve the reproducibility of the reaction. It was determined that if a sulfuric acid neutralizing aliquot of 10 N NaOH was added directly to the acidic methyl salicylate solution (e.g., after step 2), that a precipitate formed in the reaction mixture and reproducibility was reduced due to the highly exothermic reaction that occurred. Further, it was surprisingly discovered that reproducibility was substantially improved by partially neutralizing the acidic methyl salicylate solution first with a less concentrated basic solution, e.g., 5 N NaOH, followed by the addition of the more concentrated basic solution to complete the neutralization. It is further contemplated that the more concentrated NaOH aliquot may be added first but in a less than fully neutralizing amount.

Importantly, the total volume of nitrate-containing reaction mixture according to the method described herein is about 1.2 mL, which is substantially less than that disclosed in Cataldo et al. (20 mL) and the Bhandari paper (4.0 mL), and thus is an improvement in terms of volume of solution that must be disposed of.

Although calorimeters may be utilized as color measurement instruments, spectrophotometers are preferred because they are more accurate and less likely to exhibit drift. The basic components of either type of instrument are a light source, a sample illumination and viewing arrangement, a means of selecting certain wavelengths of light for the measurement, a detector of the light reflected from the sample, and some relatively simple computing capacity. The spectrophotometer or colorimeter may be handheld/transportable or a conventional laboratory instrument of varying complexity and footprint size.

The instructions provided in the assay kit for the preparation of the nitrate standard curve may read essentially as follows:

1. Transfer the aliquots of 1 mM sodium nitrate solution disclosed in Table 1 to separate glass tubes or other H₂SO₄ resistant tubes, e.g., disposable cuvettes that do not interfere with absorbance measurements at λ=410 nm, having a 10-15 mL capacity. Adjust the volume to 100 μL with distilled water as disclosed. If higher concentrations of nitrate must be determined, sample tube numbers 9 and 10 may be prepared using the 10 mM solution provided in the kit (or described in the instructions);

TABLE 1
Suggested concentrations and volumes for standard nitrate curve.

| | [nitrate] | volume | water | standard | | Approximate |
|---|---|---|---|---|---|---|
| Tube No. | supplied | (μL) | (μL) | [nitrate] | | Absorbance |
| 1 | 1 mM | 0 | 100 | 0 | | 0 |
| 2 | 1 mM | 2 | 98 | 0.02 | mM | 0.02 |
| 3 | 1 mM | 5 | 95 | 0.05 | mM | 0.044 |
| 4 | 1 mM | 10 | 90 | 0.1 | mM | 0.091 |
| 5 | 1 mM | 25 | 75 | 0.25 | mM | 0.231 |
| 6 | 1 mM | 50 | 50 | 0.50 | mM | 0.469 |
| 7 | 1 mM | 75 | 25 | 0.75 | mM | 0.69 |
| 8 | 1 mM | 100 | 0 | 1 | mM | 0.93 |
| 9 | 10 mM | 20 | 80 | 2 | mM | 1.845 |
| 10 | 10 mM | 30 | 70 | 3 | mM | 2.535 |

2. Add about 200 μL of Reagent A to each tube and vortex to mix. The time of vortexing may be from about 5 sec to about 30 sec, preferably about 10 sec. If cloudiness is observed, re-vortex until the opacity fades;
3. Add about 500 μL of Reagent B to each tube slowly and carefully vortex to mix. The time of vortexing may be from about 5 sec to about 30 sec, preferably about 10 sec. It is not abnormal for the solution to have some level of opaqueness;
4. Add about 400 μL of Reagent C to each tube slowly and carefully vortex to mix. The time of vortexing may be from about 5 sec to about 30 sec, preferably about 10 sec. If cloudiness is observed, re-vortex until the opacity fades;
5. Let each reaction mixture cool for about 5 to about 10 min; and
6. Transfer each reaction mixture to a glass cuvette and measure absorbance at λ=410 nm using a colorimeter or a spectrophotometer. Prepare the standard nitrate curve, calculate the equation for the best-fit straight line, and perform linear regression. Preferably, the square of the correlation coefficient (r²) of the best-fit straight line is greater than 0.98, more preferably greater than 0.99.

It is understood by one skilled in the art that the volume and/or concentration of the reagents used may be altered as long as the ratios of components to one another are substantially similar to those disclosed herein. For example, the volumes of the reagents used may increase or decrease depending on the amount of sample available or the degree of dilution. Alternatively, the concentration and volume of the reagents used may be altered commensurate to one another to increase or decrease the absorbance of the reaction mixture.

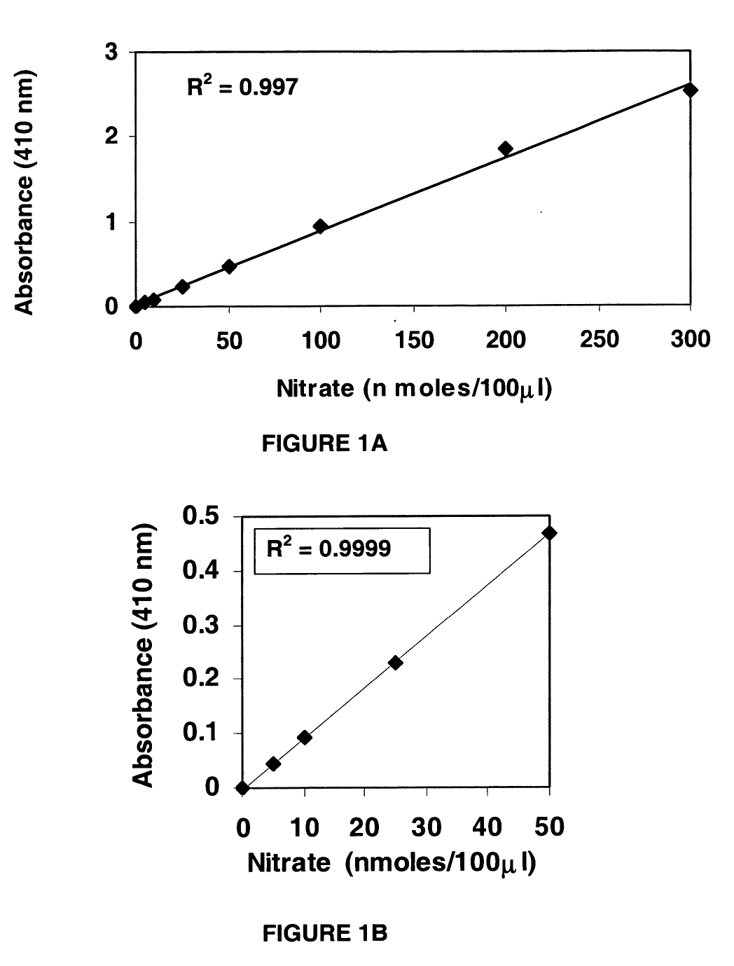

Standard nitrate curves according to the present invention are illustrated in FIGS. 1A and 1B using the absorbance data reported in Table 1. As seen in FIG. 1B, the standard nitrate curve is linear (having a correlation coefficient of 0.9999) in a range from 0 to 50 nmoles per 100 μL of solution. FIG. 1A illustrates that the standard nitrate curve is linear (having a correlation coefficient of 0.997) over a larger range from 0 to 300 nmoles per 100 μL of solution.

Additional precautions that should be included in the instructions provided in the nitrate ion assay kit include:
1. The reaction of steps 3 and 4 is exothermic so add reagents together slowly.
2. Do not cool the reaction mixture with ice. Temperatures approximating those of ice will induce precipitation of the mixture.
3. Do not transfer the reaction mixture of step 5 immediately to cold or wet cuvettes since the temperature of the cuvettes may be cool enough to induce precipitation. Preferably, the cuvettes are pre-warmed, e.g., at 37° C. for 5 min, prior to transferring the reaction mixture to the cuvette for the absorbance measurement.
4. Avoid the use of plastic cuvettes which may induce precipitation of the reaction mixture.
5. Do not use pipette tips having narrow openings during steps 2 or 6. Use wide mouth pipette tips or alternatively trim the end of narrow tips to ensure easy pipetting.
6. Reagent A is viscous so make sure that Reagent A is transferred completely from the pipet to the tube during Reagent A transference steps.
7. If no color is observed in a solution that definitively contains nitrate following the addition of Reagent C, additional drops of Reagent C may be added until the color appears. It is likely that the transference of the viscous reconstituted Reagent A resulted in a change in the concentration of H₂SO₄ in the reaction mixture.
8. If precipitation occurs during step 5, centrifuge and carefully decant the supernatant into the cuvette for the absorbance measurement. Importantly, this precipitation does not affect the absorbance of the translucent reaction mixture, i.e., the supernatant.

Comparing the sensitivity of the nitrate determination method described herein, as presented in Table 2, it can be seen that the present method is about 15.3 times more sensitive that the Cataldo method and about 3.6 times more sensitive than the method disclosed by the present inventor in the Bhandari paper.

TABLE 2
Comparison of the sensitivity of the various nitrate ion assays.

| | x nmoles of NO₃⁻ | y μg of NO₃⁻—N |
|---|---|---|
| | per 100 μL at | per 100 μL at |
| Method | an absorbance of 1 | an absorbance of 1 |
| Cataldo et al. | 1532 | 21.45 |
| Bhandari paper | 357 | 5.0 |
| this disclosure | 103 | 1.4 |

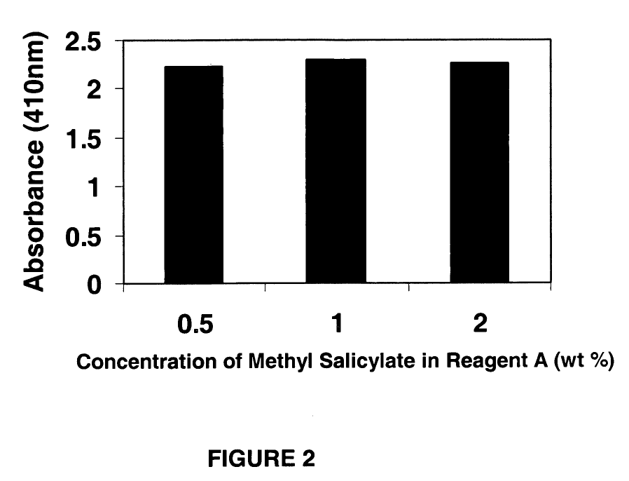

To determine the importance of the concentration of the methyl salicylate reactant, an experiment was conducted wherein the concentration of methyl salicylate in Reagent A was varied from 0.5 vol. % to 2 vol. %, based on the total volume of the solution. The reaction mixture was prepared according to the method disclosed herein, wherein the standard nitrate solution had a concentration of 250 nmoles of nitrate per 100 μL of solution. Referring to FIG. 2, which illustrates the results of this experiment, it can be seen that the concentration of methyl salicylate in Reagent A had no significant effect on the absorbance of the nitrate-containing reaction mixture (in the range investigated).

To demonstrate the ideal storage conditions of the reagents in the kit described herein, the following experiments were performed. Similar to the previous experiment, the standard nitrate solution consisted of 250 nmoles of nitrate per 100 μL of solution. To determine what effect storage has on reconstituted Reagent A, i.e., Reagent A plus concentrated sulfuric acid, a series of experiments were conducted wherein the reconstituted Reagent A was subjected to a variety of temperatures for a specific number of days and subsequently used as a reactant in the nitrate assay described herein. The results of these experiments, reported as absorbance measurements of the nitrate solution having 250 nmoles of nitrate per 100 μL of solution, are shown in Table 3.

TABLE 3
Comparison of the absorbance of a nitrate solution having
250 nmoles of nitrate per 100 μL of solution following
storage at varying conditions over time.

| | Absorbance at λ = 410 nm | | |
|---|---|---|---|
| | Room Temp. | Refrigerator | Freezer |
| Day | storage | storage | storage |
| 0 | 2.26 | 2.26 | 2.26 |
| 10 | 0.31 | 0.95 | 2.31 |
| 60 | 0.05 | 0.1 | 2.42 |
| 100 | 0.05 | 0.05 | 2.21 |

The experimental results tabulated in Table 3 illustrate that the best storage conditions for reconstituted Reagent A is at temperatures less than 0° C., most preferably about −20° C. It is noted that following removal of reconstituted Reagent A from the freezer and warming to room temperature, there was no reported change in the experimentally determined absorbance of the standard nitrate solution consisting of 250 nmoles of nitrate per 100 μL of solution. The storage temperature of the reagent was a significant limitation of the Cataldo et al. method, which translated to reproducibility problems.

Reagents that may potentially be used to replace methyl salicylate in Reagent A of the kit described herein include, but are not limited to, phenol, p-cresol, benzoic acid, 4-methyl benzoic acid, p-hydroxy benzoic acid and acetylsalicylic acid. Alternatively, derivatives of methyl salicylate having a light producing group may be used whereby nitrate concentrations are quantified luminescently using a luminometer.

Prior to measurement for nitrate ions, the sample solutions should be substantially devoid of proteinaceous material. Towards that end, proteins in the sample solution may be removed using any commercially available chromatography columns, or equivalent thereof, as readily determined by one skilled in the art.

Another embodiment of the nitrate assay kit includes a well plate method to allow the user to quantify multiple samples simultaneously. The well plate kit may optionally include a well plate. The instructions provided in the assay kit for the determination of nitrate ion concentration may read essentially as follows:
1. Transfer about 0-20 μL of a sample solution containing nitrate (0-300 nmole or 0-4.2 μg NO₃⁻—N) to a well on a 96 well plate. Adjust the volume to 20 μL with distilled water, if necessary;
2. Add about 40 μL of Reagent A to the well and mix, e.g., tapping the well by hand or equivalent thereof;
3. Let the plate stand for approximately 5 minutes to allow the solutions to properly mix;
4. Add about 100 μL of Reagent B to the well and mix;
4. Add about 100 μL of Reagent C to the well and mix;
5. Let the reaction mixture cool for about 5 to about 10 min; and
6. Measure the absorbance of the complex at λ=405-410 nm using a plate reader. Using Beer's Law and the standard nitrate curve, calculate the concentration of nitrate in the sample.

It is understood by one skilled in the art that the volume and/or concentration of the reagents used may be altered as long as the ratios of components to one another are substantially similar to those disclosed herein. For example, the volumes of the reagents used may increase or decrease depending on the amount of sample available or the degree of dilution. Alternatively, the concentration and volume of the reagents used may be altered commensurate to one another to increase or decrease the absorbance of the reaction mixture.

Preparation of the standard nitrate curve for the well plate nitrate determination method is well within the knowledge of one skilled in the art.

In yet another embodiment, the assay kit for conducting nitrate assays described herein may further include a method for directly determining the concentration of nitrite in a sample solution. The micro-method determination for nitrite is a derivative of the well-known Greiss diazotization reaction. Under highly acidic conditions, sulfanilamide or sulfanilic acid (the diazotization reagents) and N-1-napthylethylene diamine hydrochloride (the coupling reagent) react with nitrite ions in the presence of hydrochloric acid to form a red azo complex having a maximum absorbance between λ=520-565 nm.

In a further embodiment, the nitrite determination method is provided in the form of a kit. Kit components include an acidic sulfanilamide solution, dilute N-1-napthylethylenediamine hydrochloride, zinc sulfate, and instructions. The kit may optionally include a well plate, and a standard solution of sodium nitrite. Similar to the nitrate determination method, the volume of reagents provided in the kit is dependent on whether the absorbance measurement is performed using a well plate reader or a spectrophotometer/colorimeter (to be discussed at length hereinbelow).

For example, the kit for 100 nitrite assays using a well plate reader may comprise the reagents in the following quantities and concentrations:
1. Standard solution of sodium nitrite: 0.5 mL of 1 mM;
2. Reagent A: 1.5 mL of 2.5 wt % sulfanilamide in 5 N HCl, based on the total weight of the solution. Reagent A is preferably stored in an amber-colored reagent bottle;
3. Reagent B: 1.5 mL of 0.04 wt % N-1-napthylethylenediamine hydrochloride in distilled water, based on the total weight of the solution. Reagent B is preferably stored in an amber-colored reagent bottle; and
4. Reagent C: 2 mL of 40 wt % zinc sulfate in distilled water, based on the total weight of the solution.
   A 96 well plate may be used to assay upwards of 96 samples having volumes up to 0.2 mL.

It is understood by one skilled in the art that the volume and/or concentration of the kit components may be altered as long as the ratios of components to one another are substantially similar to those disclosed herein. For example, the volumes of the kit components may increase or decrease depending on the number of assays desired. Alternatively, the concentration and volume of the kit components may be altered commensurate to one another to increase or decrease the volume of each individual assay.

The instructions provided in the assay kit for the determination of nitrite ion concentration may read essentially as follows:
1. If the solution contains proteins, go to step 2. If the solution does not contain proteins, transfer about 0-200 μL of a sample solution to a well on the 96 well plate. Adjust the volume to 200 μL with distilled water, if necessary. Go to step 3;
2. If the solution contains proteins, which are likely to interfere with the Greiss reaction, transfer about 0-200 μL of a solution to a tube having a volume of at least 1.5 mL. The tube may be any centrifuge tube resistant to high concentrations of HCl, e.g., Eppendorf tubes. Adjust the volume to 200 μL with distilled water, if necessary. Add 20 μL of Reagent C to the tube and incubate at room temperature for about 5 minutes. Following incubation, the mixture must be centrifuged to separate the supernatant (comprising the nitrite ions) from the solids (comprising the proteins). Transfer the supernatant to a well on the 96 well plate.
3. Add 15 μL of Reagent A to the well containing the solution to be analyzed;
4. Add 15 μL of Reagent B to the well containing the solution to be analyzed. Mix contents;
5. Let each reaction mixture develop for about 15 min; and
6. Measure the absorbance of the azo complex at λ=550 nm using a plate reader. Using Beer's Law and the standard nitrite curve, calculate the concentration of nitrite in the sample.

It is understood by one skilled in the art that the volume and/or concentration of the reagents used may be altered as long as the ratios of components to one another are substantially similar to those disclosed herein. For example, the volumes of the reagents used may increase or decrease depending on the amount of sample available or the degree of dilution. Alternatively, the concentration and volume of the reagents used may be altered commensurate to one another to increase or decrease the absorbance of the reaction mixture.

The instructions provided in the assay kit for the preparation of the nitrite standard curve may read essentially as follows:

1. Transfer the aliquots of 1 mM sodium nitrite solution disclosed in Table 4 to separate wells on the well plate. Adjust the volume to 200 μL with distilled water as disclosed;

TABLE 4
Suggested concentrations and volumes for standard nitrite curve.

| | | volume | | | | |
|---|---|---|---|---|---|---|
| Tube | nitrite | 1 mM NO₂⁻ | water | standard | | Approximate |
| No. | (nmoles) | delivered (μL) | (μL) | [nitrite] | | Absorbance |
| 1 | 0 | 0 | 200 | 0 | | 0 |
| 2 | 1 | 1 | 199 | 0.005 | mM | 0.09 |
| 3 | 2 | 2 | 198 | 0.01 | mM | 0.16 |
| 4 | 5 | 5 | 195 | 0.025 | mM | 0.48 |
| 5 | 7.5 | 7.5 | 192.5 | 0.038 | mM | 0.71 |
| 6 | 10 | 10 | 190 | 0.05 | mM | 0.92 |
| 7 | 12.5 | 12.5 | 187.5 | 0.063 | mM | 1.36 |
| 8 | 15 | 15 | 185 | 0.075 | mM | 1.58 |
| 9 | 20 | 20 | 180 | 0.1 | mM | 2.15 |

2. Add 15 μL of Reagent A to the well containing the solution to be analyzed;
3. Add 15 μL of Reagent B to the well containing the solution to be analyzed. Mix contents;
4. Let each reaction mixture develop for about 15 min; and
5. Measure the absorbance of the azo complex at λ=550 nm using a plate reader. Prepare the standard nitrite curve, calculate the equation for the best-fit straight line, and perform linear regression. Preferably, the square of the correlation coefficient (r²) of the best-fit straight line is greater than 0.98, more preferably greater than 0.99.

It is understood by one skilled in the art that the volume and/or concentration of the reagents used may be altered as long as the ratios of components to one another are substantially similar to those disclosed herein. For example, the volumes of the reagents used may increase or decrease depending on the amount of sample available or the degree of dilution. Alternatively, the concentration and volume of the reagents used may be altered commensurate to one another to increase or decrease the absorbance of the reaction mixture.

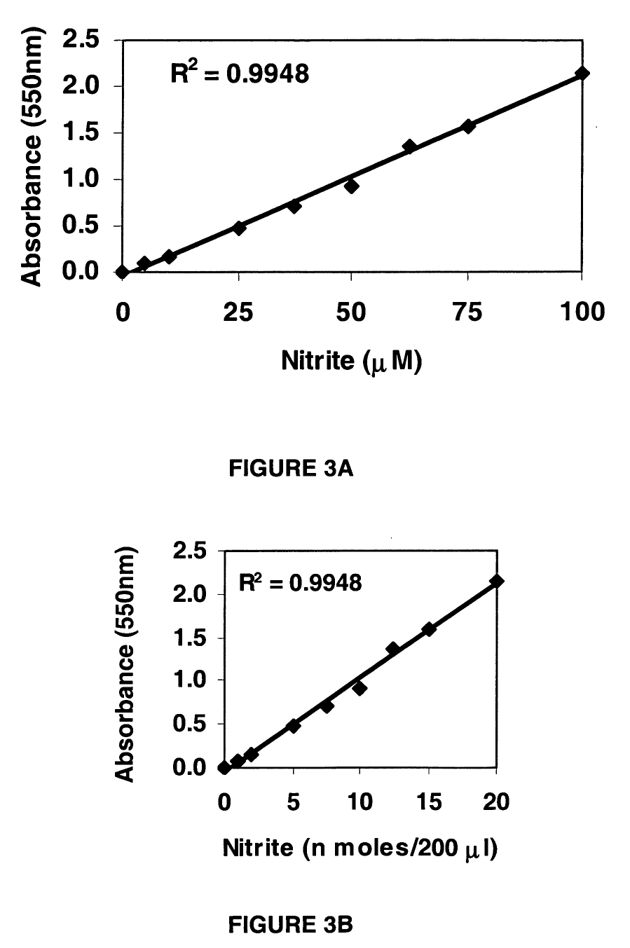

Standard nitrite curves according to the present invention are illustrated in FIGS. 3A and 3B using the absorbance data reported in Table 4. As seen in FIG. 3B, the standard nitrite curve is linear (having a correlation coefficient of 0.9948) in a range from 0 to 20 nmoles per 200 μL of solution. FIG. 3A illustrates that the standard nitrite curve is linear (having a correlation coefficient of 0.9948) in a range from 0 to 100 μm nitrite.

Another embodiment of the nitrite assay kit for spectrophotometric determination may comprise the reagents in the following quantities and concentrations:
1. Standard solution of sodium nitrite: 10 mL of 0.1 mM;
2. Reagent A: 7.5 mL of 2.5 wt % sulfanilamide in 5 N HCl, based on the total weight of the solution. Reagent A is preferably stored in an amber-colored reagent bottle;
3. Reagent B: 7.5 mL of 0.04 wt % N-1-napthylethylenediamine hydrochloride in distilled water, based on the total weight of the solution. Reagent B is preferably stored in an amber-colored reagent bottle; and
4. Reagent C: 10 mL of 40 wt % zinc sulfate in distilled water, based on the total weight of the solution.

It is understood by one skilled in the art that the volume and/or concentration of the kit components may be altered as long as the ratios of components to one another are substantially similar to those disclosed herein. For example, the volumes of the kit components may increase or decrease depending on the number of assays desired. Alternatively, the concentration and volume of the kit components may be altered commensurate to one another to increase or decrease the volume of each individual assay.

The instructions provided in the spectrophotometric assay kit for the determination of nitrite ion concentration may read essentially as follows:
1. If the solution contains proteins, go to step 2. If the solution does not contain proteins, transfer about 0-0.9 mL of a sample solution to a tube having a volume greater than 1 mL, preferably about 1.5 mL. The tube may be any centrifuge tube resistant to high concentrations of HCl, e.g., Eppendorf tubes, that do not interfere with absorbance measurements at λ=500-550 nm. Adjust the volume to 0.9 mL with distilled water, if necessary. Go to step 3;
2. If the solution contains proteins, which are likely to interfere with the Greiss reaction, transfer about 0-0.9 mL of a solution to a tube having a volume of at least 1.5 mL. The tube may be any tube resistant to high concentrations of HCl that do not interfere with absorbance measurements at λ=500-550 nm. Adjust the volume to 0.9 μL with distilled water, if necessary. Add 90-100 μL of Reagent C to the tube and incubate at room temperature for about 5 minutes. Following incubation, the mixture must be centrifuged to separate the supernatant (comprising the nitrite ions) from the solids (comprising the proteins).
3. Add 75 μL of Reagent A to the tube containing the solution to be analyzed;
4. Add 75 μL of Reagent B to the tube containing the solution to be analyzed. Mix contents;
5. Let each reaction mixture develop for about 15 min; and
6. Measure the absorbance of the azo complex at λ=550 nm using a spectrophotometer. Using Beer's Law and the standard nitrite curve, calculate the concentration of nitrite in the sample.

It is understood by one skilled in the art that the volume and/or concentration of the reagents may be altered as long as the ratios of components to one another are substantially similar to those disclosed herein. For example, the volumes of the reagents used may increase or decrease depending on the amount of sample available or the degree of dilution. Alternatively, the concentration and volume of the reagents used may be altered commensurate to one another to increase or decrease the absorbance of the reaction mixture.

The instructions provided in the photometric assay kit for the preparation of the nitrite standard curve may read essentially as follows:

1. Transfer the aliquots of 1 mM sodium nitrite solution disclosed in Table 5 to separate tubes having a volume greater than 1 mL, preferably about 1.5 mL. The tube may be any tube resistant to high concentrations of HCl that do not interfere with absorbance measurements at λ=500-550 nm. Adjust the volume to 0.9 mL with distilled water as disclosed;

TABLE 5
Suggested concentrations and volumes for standard nitrite curve.

| | | volume | | | | |
|---|---|---|---|---|---|---|
| Tube | nitrite | 1 mM NO₂⁻ | water | standard | | Approximate |
| No. | (nmoles) | delivered (μL) | (μL) | [nitrite] | | Absorbance |
| 1 | 0 | 0 | 200 | 0 | | 0 |
| 2 | 1 | 1 | 199 | 0.005 | mM | 0.04 |
| 3 | 3 | 3 | 197 | 0.015 | mM | 0.14 |
| 4 | 5 | 5 | 195 | 0.025 | mM | 0.22 |
| 5 | 10 | 10 | 190 | 0.05 | mM | 0.43 |
| 6 | 20 | 20 | 180 | 0.1 | mM | 0.80 |
| 7 | 30 | 30 | 170 | 0.15 | mM | 1.15 |

2. Add 75 μL of Reagent A to the tube containing the solution to be analyzed;
3. Add 75 μL of Reagent B to the tube containing the solution to be analyzed. Mix contents;
4. Let each reaction mixture develop for about 15 min; and
5. Measure the absorbance of the azo complex at λ=550 nm using a spectrophotometer. Prepare the standard nitrite curve, calculate the equation for the best-fit straight line, and perform linear regression. Preferably, the square of the correlation coefficient (r²) of the best-fit straight line is greater than 0.98, more preferably greater than 0.99.

It is understood by one skilled in the art that the volume and/or concentration of the reagents used may be altered as long as the ratios of components to one another are substantially similar to those disclosed herein. For example, the volumes of the reagents used may increase or decrease depending on the amount of sample available or the degree of dilution. Alternatively, the concentration and volume of the reagents used may be altered commensurate to one another to increase or decrease the absorbance of the reaction mixture.

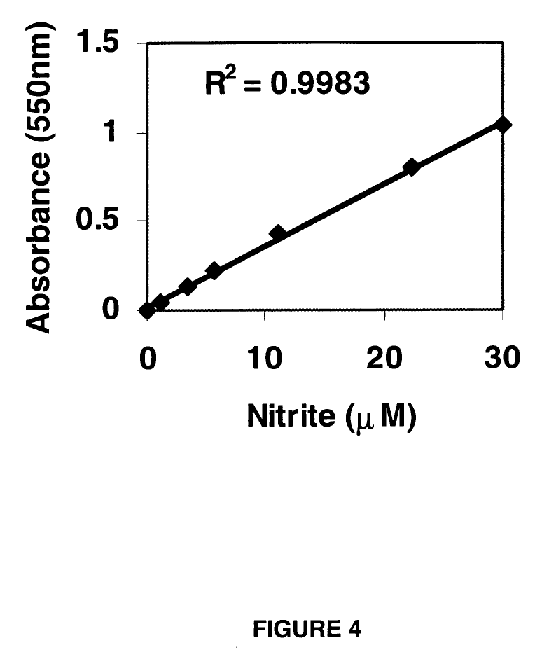

A standard nitrite curve according to the present invention is illustrated in FIG. 4 using the absorbance data reported in Table 5. As seen in FIG. 4, the standard nitrite curve is linear (having a correlation coefficient of 0.9983) in a range from 0 to 30 μM.

It is to be appreciated by the skilled artisan that the present invention relates to an assay kit and method consisting of a nitrate assay kit described herein, or an equivalent thereof, or in the alternative, the present invention relates to an assay kit and method comprising a nitrate assay kit described herein, or an equivalent thereof, and a nitrite assay kit described herein, or equivalent thereof.

Furthermore, the present invention is advantageous over prior art assay kits and methods because it provides the reagents and instructions to simultaneously and directly determine the concentration of nitrate and nitrite ions in a simple, rapid, reproducible manner without the need to purchase new and expensive equipment.

It is to be appreciated by one skilled in the art that the reagents, methods and kits disclosed herein may be altered for the manual or automatic, e.g., robotic, determination of nitrate or nitrate and nitrite ions in a sample solution.

In yet another embodiment of the present invention, nitrate concentrations may be approximated using testing strips embedded with the appropriate reagents. Specifically, a testing strip may have one or more of the testing reagents described hereinabove embedded on it, e.g., sulfuric acid, and the testing strip is sequentially dipped in the sample solution, a methyl salicylate solution, and a NaOH solution, whereby the presence of nitrate ions in the sample solution triggers a colorimetric change on the testing strip which may be compared with a color chart to approximate the concentration of nitrate ions in the sample solution.

For example, the color chart can signify a variety of color changes depending on the concentration range of nitrate ions in the sample solution, analogous to a litmus paper pH range. Alternatively, the testing strip may be specific to a concentration, e.g., a nitrate concentration indicative of early onset diabetes, heart disease, cancer, etc. In this latter embodiment, only one color change is necessary to indicate that the concentration of nitrate in the sample solution is in an unhealthy range.

The testing strip may be manufactured from paper, plastic or silica-based substances, with the qualification that at least one of the necessary reagents may be embedded in the testing strip without alteration of the concentration of the reagents embedded in the strip and/or interference of the testing strip material with the chemical reaction.

Accordingly, although the invention has been variously disclosed herein with reference to illustrative aspects, embodiments and features, it will be appreciated that the aspects, embodiments and features described hereinabove are not intended to limit the invention, and that other variations, modifications and other embodiments will suggest themselves to those of ordinary skill in the art. The invention therefore is to be broadly construed, consistent with the claims hereafter set forth.

What is claimed is:

1. An assay kit comprising reagents for conducting a nitrate assay, wherein when said reagents are combined with a nitrate-containing sample, a colorimetric change is provided which is detectable by spectrophotometry or colorimetry and enables the concentration of nitrate in the nitrate-containing sample to be directly determined.

2. The assay kit of claim 1, wherein the nitrate assay reagents comprise:
(a) a methyl salicylate solution; and
(b) at least one sodium hydroxide solution having a pH greater than 14.

3. The assay kit of claim 2, wherein the concentration of methyl salicylate in said solution is in a range of about 0.5 vol. % to about 2 vol. %, based on the total volume of solution.

4. The assay kit of claim 2, further comprising a second sodium hydroxide solution having a pH that is numerically greater than the pH of a first sodium hydroxide solution.

5. The assay kit of claim 4, wherein the pH of the first sodium hydroxide solution is in a range of about 2 M to about 8 M.

6. The assay kit of claim 4, wherein the pH of the first sodium hydroxide solution is in a range of about 4 M to about 6 M.

7. The assay kit of claim 4, wherein the pH of the second sodium hydroxide solution is in a range of about 6 M to about 12 M.

8. The assay kit of claim 2, further comprising sulfuric acid.

9. The assay kit of claim 8, wherein the concentration of sulfuric acid is in a range from about 15 M to about 18 M.

10. The assay kit of claim 2, further comprising at least one standard solution of sodium nitrate.

11. The assay kit of claim 2, further comprising at least one tube having volume capacity in a range of about 1 mL to about 15 mL.

12. The assay kit of claim 2, further comprising instructions for conducting the nitrate assay.

13. The assay kit of claim 12, wherein the instructions comprise:
(a) transferring an aliquot of the nitrate-containing sample to a tube;
(b) mixing an aliquot of sulfuric acid with an aliquot of the methyl salicylate solution to form an acidic methyl salicylate solution;
(c) adding an aliquot of the acidic methyl salicylate solution to the nitrate-containing sample to form an acidic reaction mixture;
(d) adding at least one aliquot of the sodium hydroxide solution to the acidic reaction mixture to form a basic reaction mixture;
(e) cooling the basic reaction mixture; and
(f) measuring the absorbance of the basic reaction mixture at λ=410 nm.

14. The assay kit of claim 13, wherein the instructions further comprise the preparation of a standard nitrate curve.

15. The assay kit of claim 13, wherein the instructions further comprise vortexing to mix the solutions in steps (b)-(d).

16. The assay kit of claim 13, wherein the concentration of sulfuric acid is in a range from about 15 M to 18 M.

17. The assay kit of claim 13, wherein the instructions further comprise the addition of a second aliquot of sodium hydroxide solution to the basic reaction mixture prior to step (e), wherein the second aliquot of sodium hydroxide has a pH that is numerically greater than the pH of a first aliquot of sodium hydroxide solution.

18. A method for directly determining the concentration of nitrate ions in a nitrate-containing sample, said method comprising:
(a) transferring an aliquot of the nitrate-containing sample to a tube;
(b) mixing an aliquot of sulfuric acid with an aliquot of a methyl salicylate solution to form an acidic methyl salicylate solution;
(c) adding an aliquot of the acidic methyl salicylate solution to the nitrate-containing sample to form an acidic reaction mixture;
(d) adding at least one aliquot of a sodium hydroxide solution to the acidic reaction mixture to form a basic reaction mixture;
(e) cooling the basic reaction mixture; and
(f) measuring the absorbance of the basic reaction mixture at λ=410 nm.

19. The method of claim 18, wherein the concentration of methyl salicylate is in a range of about 0.5 vol. % to about 2 vol. %, based on the total volume of solution.

20. The method of claim 18, further comprising the preparation of a standard nitrate curve.

21. The method of claim 18, wherein the absorbance is measured using an analytical process selected from the group consisting of spectrophotometry and colorimetry.

22. The method of claim 18, further comprising vortexing to mix the solutions in steps (b)-(d).

23. The method of claim 18, wherein the concentration of sulfuric acid is in a range from about 15 M to about 18 M.

24. The method of claim 18, further comprising the addition of a second aliquot of sodium hydroxide solution to the basic reaction mixture prior to step (e), wherein the second aliquot of sodium hydroxide has a pH that is numerically greater than the pH of a first aliquot of sodium hydroxide solution.

25. The method of claim 24, wherein the pH of the first aliquot of sodium hydroxide solution is in a range of about 2 M to about 8 M.

26. The method of claim 24, wherein the pH of the first aliquot of sodium hydroxide solution is in a range of about 4 M to about 6 M.

27. The method of claim 24, wherein the pH of the second aliquot of sodium hydroxide solution is in a range of about 6 M to about 12 M.

28. An assay kit comprising reagents for conducting a nitrate assay and a nitrite assay, wherein when said reagents are combined with a sample solution, a calorimetric change is provided which is detectable spectrophotometrically or colorimetry, and enables the concentration of nitrate and nitrite in a sample solution to be separately and directly determined.

29. The assay kit of claim 28, wherein the nitrate assay reagents comprise:
(a) a methyl salicylate solution; and
(b) at least one sodium hydroxide solution having a pH greater than 14.
and wherein the nitrite assay reagents comprise:
(a) a sulfanilimide solution;
(b) a N-1-napthylethylenediamine hydrochloride solution; and
(c) a zinc sulfate solution.

30. The assay kit of claim 29, wherein the concentration of methyl salicylate in said solution is in a range of about 0.5 vol. % to about 2 vol. %, based on the total volume of solution.

31. The assay kit of claim 29, further comprising a second sodium hydroxide solution having a pH that is numerically greater than the pH of a first sodium hydroxide solution.

32. The assay kit of claim 31, wherein the pH of the first sodium hydroxide solution is in a range of about 2 M to about 8 M.

33. The assay kit of claim 31, wherein the pH of the first sodium hydroxide solution is in a range of about 4 M to about 6 M.

34. The assay kit of claim 31, wherein the pH of the second sodium hydroxide solution is in a range of about 8 M to about 12 M.

35. The assay kit of claim 29, further comprising sulfuric acid for the nitrate assay.

36. The assay kit of claim 35, wherein the concentration of sulfuric acid is in a range from about 15 M to about 18 M.

37. The assay kit of claim 29, further comprising at least one standard solution selected from the group consisting of a sodium nitrate standard solution and a sodium nitrite standard solution.

38. The assay kit of claim 29, further comprising instructions for conducting the nitrate assay and the nitrite assay.
